(19) United States
(12) Patent Application Publication
(10) Pub. No.: US 2026/0248403 A1
(43) Pub. Date: Aug. 27, 2026

(54) METHOD AND APPARATUS FOR ANALYZING WORK AND HEALTH STATUS OF USER IN OFFICE USING RADAR

(71) Applicant: SEWON INTELLIGENCE Co., Ltd, Seoul (KR)

(72) Inventors: Se-jin PARK, Anseong-si (KR); Ha-na JEONG, Seoul (KR); Young SEO, Daejeon (KR)

(73) Assignee: SEWON INTELLIGENCE Co., Ltd, Seoul (KR)

(21) Appl. No.: 19/060,896

(22) Filed: Feb. 24, 2025

(51) Int. Cl. A61B 5/05; G01S 7/41; G01S 13/88; G16H 50/30; G16H 70/20

(52) U.S. Cl. CPC A61B 5/05; G01S 7/415; G01S 13/88; G16H 50/30; G16H 70/20

(57) ABSTRACT

Disclosed is a method and apparatus for analyzing a work and health status of user in an office using radar. In an aspect of the present disclosure, an apparatus for analyzing a work and health status using radar according to at least one of the embodiments may include a memory; a radar sensor positioned in front of a user to sense movement data of the user while working; and a processor configured to analyze the work and health status by obtaining data sensed by the radar sensor, wherein the processor is configured to convert the data sensed by the radar sensor into a frequency domain and obtain respiratory activity waveform and heartbeat waveform data.

### BACKGROUND

### 1. Technical Field

The present disclosure relates to a method and apparatus for analyzing a work and health status of a user in an office using radar.

### 2. Description of Related Art

People spend a considerable amount of time in an office each day. Since users are physically or mentally engaged in labor in the office, the probability of health-related problems occurring is relatively higher than when they spend time at home.

Therefore, by identifying the status of users working in the office and responding appropriately, problems can be prevented in advance or bigger problems can be prevented. Therefore, in such situations, it is necessary to devise a method to accurately identify the work and health status of users.

### SUMMARY

The purpose of the embodiment disclosed in the present disclosure is to provide a method and apparatus that can more accurately identify and analyze the work and health status of a user located in an office and respond to it by using a radar sensor.

Technical problems of the inventive concept are not limited to the technical problems mentioned above, and other technical problems not mentioned will be clearly understood by those skilled in the art from the following description.

In an aspect of the present disclosure, an apparatus for analyzing a work and health status using radar according to at least one of the embodiments may include a memory; a radar sensor positioned in front of a user to sense movement data of the user while working; and a processor configured to analyze the work and health status by obtaining data sensed by the radar sensor, wherein the processor is configured to convert the data sensed by the radar sensor into a frequency domain and obtain respiratory activity waveform and heartbeat waveform data.

In another aspect of the present disclosure, a method for analyzing a work and health status using radar disposed in front of each user performed by a processor of an electronic apparatus according to at least one of the embodiments may include storing reference data; obtaining movement data of a user while working using the radar; converting the obtained movement data of the user while working into a frequency domain and obtaining respiratory activity waveform and heartbeat waveform data; analyzing the obtained respiratory and heartbeat waveform data by comparing the data with the stored reference data; and generating and transmitting guide data including recommended content based on the analysis result.

Other specific details of the present disclosure are included in the detailed description and drawings.

### BRIEF DESCRIPTION OF THE FIGURES

FIG. 1 is a diagram illustrating a system according to an embodiment of the present disclosure.

FIG. 2 is a drawing illustrating the processor of FIG. 1.

FIGS. 3A to 4B are diagrams illustrating an arrangement of the radar in the workspace in the office.

FIGS. 5 and 6 are flowcharts illustrating a method for analyzing a user's work status using radar according to an embodiment of the present disclosure.

FIGS. 7A and 7B illustrate examples of a guide according to an embodiment of the present disclosure.

### DETAILED DESCRIPTION

In the drawings, the same reference numeral refers to the same element. This disclosure does not describe all elements of embodiments, and general contents in the technical field to which the present disclosure belongs or repeated contents of the embodiments will be omitted. The terms, such as “unit, module, member, and block” may be embodied as hardware or software, and a plurality of “units, modules, members, and blocks” may be implemented as one element, or a unit, a module, a member, or a block may include a plurality of elements.

Throughout this specification, when a part is referred to as being “connected” to another part, this includes “direct connection” and “indirect connection”, and the indirect connection may include connection via a wireless communication network.

Furthermore, when a certain part “includes” a certain element, other elements are not excluded unless explicitly described otherwise, and other elements may in fact be included.

In the entire specification of the present disclosure, when any member is located “on” another member, this includes a case in which still another member is present between both members as well as a case in which one member is in contact with another member.

The terms “first,” “second,” and the like are just to distinguish an element from any other element, and elements are not limited by the terms.

The singular form of the elements may be understood into the plural form unless otherwise specifically stated in the context.

Identification codes in each operation are used not for describing the order of the operations but for convenience of description, and the operations may be implemented differently from the order described unless there is a specific order explicitly described in the context.

The operating principle and embodiments of the present disclosure are described below with reference to the attached drawings.

In this specification, the term ‘apparatus according to the present disclosure’ includes all of various devices that can perform computational processing and provide results to the user. For example, the device may include all of a computer, a server device, and a portable terminal, or may be in the form of one of them.

Here, the computer may include, for example, a notebook, a desktop, a laptop, a tablet PC, a slate PC, and the like mounted with a web browser.

The server device is a server that communicates with an external device to process information, and may include an application server, a computing server, a database server, a file server, a mail server, a proxy server, and a web server.

A portable terminal is a wireless communication device that ensures portability and mobility, and may include all kinds of handheld-based wireless communication devices such as PCS (Personal Communication System), GSM (Global System for Mobile communications), PDC (Personal Digital Cellular), PHS (Personal Handyphone System), PDA (Personal Digital Assistant), IMT (International Mobile Telecommunication)-2000, CDMA (Code Division Multiple Access)-2000, W-CDMA (W-Code Division Multiple Access), WiBro (Wireless Broadband Internet) terminal, a smart phone, and the like, and a wearable device such as at least one of a watch, a ring, bracelets, anklets, a necklace, glasses, contact lenses, or a head-mounted device (HMD).

The function related to artificial intelligence according to the present disclosure operates through a processor and a memory. The processor may be composed of one or more processors. At this time, the one or more processors may be a general-purpose processor such as a CPU, an AP, a DSP (Digital Signal Processor), a graphics-only processor such as a GPU, a VPU (Vision Processing Unit), or an artificial intelligence-only processor such as an NPU. The one or more processors control input data to be processed according to a predefined operation rule or artificial intelligence model stored in the memory. Alternatively, in the case that the one or more processors are artificial intelligence-only processors, the artificial intelligence-only processor may be designed as a hardware structure specialized for processing a specific artificial intelligence model.

The predefined operation rule or artificial intelligence model may be created through learning. Here, being created through learning means that a basic artificial intelligence model is learned by using a plurality of learning data by a learning algorithm, thereby creating a predefined operation rule or artificial intelligence model set to perform a desired characteristic (or, purpose). Such learning may be performed on the device itself in which the artificial intelligence according to the present disclosure is performed, or may be performed through a separate server and/or system. Examples of learning algorithms include supervised learning, unsupervised learning, semi-supervised learning, or reinforcement learning, but are not limited to the examples described above.

The artificial intelligence model may include a plurality of neural network layers. Each of the plurality of neural network layers has a plurality of weights, and performs neural network operations through operations between the operation results of the previous layer and the plurality of weights. The plurality of weights of the plurality of neural network layers may be optimized by the learning results of the artificial intelligence model. For example, the plurality of weights may be updated so that the loss value or cost value acquired by the artificial intelligence model is reduced or minimized during the learning process. The artificial neural network may include a deep neural network (DNN), for example, a convolutional neural network (CNN), a deep neural network (DNN), a recurrent neural network (RNN), a restricted Boltzmann machine (RBM), a deep belief network (DBN), a bidirectional recurrent deep neural network (BRDNN), or a deep Q-network, but is not limited to the examples described above.

According to an exemplary embodiment of the present disclosure, the processor may implement artificial intelligence. Artificial intelligence refers to a machine learning method based on an artificial neural network that imitates human neurons (biological neurons) to enable a machine to learn. The artificial intelligence methodology may be divided into supervised learning in which input data and output data are provided together as training data according to a learning method so that the answer (output data) to a problem (input data) is determined, unsupervised learning in which only input data is provided without output data so that the answer (output data) to a problem (input data) is not determined, and reinforcement learning in which a reward is given from an external environment whenever an action is taken in a current state (State), and learning is performed in a direction to maximize this reward. In addition, the methodology of artificial intelligence can be classified according to the architecture, which is the structure of the learning model. The architecture of widely used deep learning technology can be classified into convolutional neural networks (CNNs), recurrent neural networks (RNNs), transformers, and generative adversarial networks (GANs).

The present device and system may include an artificial intelligence model. The artificial intelligence model may be one artificial intelligence model or may be implemented as multiple artificial intelligence models. The artificial intelligence model may be composed of a neural network (or artificial neural network) and may include a statistical learning algorithm that mimics the neurons of biology in machine learning and cognitive science. A neural network may mean an overall model that has problem-solving capabilities by changing the strength of the synapse connection through learning by forming a network with artificial neurons (nodes) that combine synapses. The neurons of the neural network may include a combination of weights or biases. The neural network may include one or more layers composed of one or more neurons or nodes. For example, the device may include an input layer, a hidden layer, and an output layer. The neural network constituting the device can infer a desired result (output) from an arbitrary input (input) by changing the weights of neurons through learning.

The processor may generate a neural network, train (or learn) a neural network, perform a calculation based on received input data, generate an information signal based on the result of the calculation, or retrain the neural network. The models of the neural network may include various types of models such as CNN (Convolution Neural Network) such as GoogleNet, AlexNet, VGG Network, R-CNN (Region with Convolution Neural Network), RPN (Region Proposal Network), RNN (Recurrent Neural Network), S-DNN (Stacking-based deep Neural Network), S-SDNN (State-Space Dynamic Neural Network), Deconvolution Network, DBN (Deep Belief Network), RBM (Restrcted Boltzman Machine), Fully Convolutional Network, LSTM (Long Short-Term Memory) Network, Classification Network, and the like, but are not limited thereto. The processor may include one or more processors for performing calculations according to the models of the neural network. For example, a neural network may include a deep neural network.

The neural network may include CNN (Convolutional Neural Network), RNN (Recurrent Neural Network), percept, multilayer perceptron, FF (Feed Forward), RBF (Radial Basis Network), DFF (Deep Feed Forward), LSTM (Long Short Term Memory), Gated Recurrent Unit (GRU), Auto Encoder (AE), Variational Auto Encoder (VAE), Denoising Auto Encoder (DAE), Sparse Auto Encoder (SAE), Markov Chain (MC), Hopfield Network (HN), Boltzmann Machine (BM), Restricted Boltzmann Machine (RBM), Depp Belief Network (DBN), Deep Convolutional Network (DCN), Deconvolutional Network (DN), Deep Convolutional Inverse Graphics Network (DCIGN), Generative Adversarial Network (GAN), Liquid State Machine (LSM), Extreme Learning Machine (ELM), Echo State Network (ESN), Deep Residual Network (DRN), Differentiable Neural Computer (DNC), Neural Turning Machine (NTM), Capsule Network (CN), Kohonen Network (KN), and Attention Network (AN), but not limited thereto, and it will be understood by those skilled in the art that any neural network may be included.

According to an exemplary embodiment of the present disclosure, the processor may use various artificial intelligence structures and algorithms such as CNN (Convolution Neural Network), R-CNN (Region with Convolution Neural Network), RPN (Region Proposal Network), RNN (Recurrent Neural Network), S-DNN (Stacking-based deep Neural Network), S-SDNN (State-Space Dynamic Neural Network), Deconvolution Network, DBN (Deep Belief Network), RBM (Restricted Boltzmann Machine), Fully Convolutional Network, LSTM (Long Short-Term Memory) Network, Classification Network, Generative Modeling, eXplainable AI, Continual AI, Representation Learning, and AI for Material Design such as GoogleNet, AlexNet, VGG Network, BERT, SP-BERT, MRC/QA, Text Analysis, Dialog System, GPT-3, and GPT-4 for natural language processing, Visual Analytics, Visual Understanding, Video Synthesis for vision processing, Anomaly Detection, Prediction, Time-Series Forecasting, Optimization, and Recommendation for algorithms ResNet for data intelligence, but not limited thereto.

The following disclosure discloses a method and apparatus for analyzing a work and health status of a user in an office using radar according to the present disclosure.

The apparatus according to the present disclosure in this specification may include various devices that may perform computational processing and provide results to a client. For example, the apparatus according to the present disclosure may include at least one computer or computing device, a server device, a terminal, and the like, or may be in the form of one of them.

In the above description, the computer may include, for example, a notebook, desktop, laptop, tablet PC, slate PC, and the like provided with a web browser.

The server device is a server that communicates with an external device to process information, and may include an application server, a computing server, a database server, a file server, a mail server, a proxy server, and a web server.

A portable terminal is a wireless communication device that ensures portability and mobility, and may include all kinds of handheld-based wireless communication devices such as PCS (Personal Communication System), GSM (Global System for Mobile communications), PDC (Personal Digital Cellular), PHS (Personal Handyphone System), PDA (Personal Digital Assistant), IMT (International Mobile Telecommunication)-2000, CDMA (Code Division Multiple Access)-2000, W-CDMA (W-Code Division Multiple Access), WiBro (Wireless Broadband Internet) terminal, a smart phone, and the like, and a wearable device such as at least one of a watch, a ring, bracelets, anklets, a necklace, glasses, contact lenses, or a head-mounted device (HMD).

In this specification, a control model may be defined or a related platform may be built in relation to a device according to the present disclosure, which may be generated and provided by a computer based on big data and artificial intelligence technology, and may be implemented using or referencing ICT (Information and Communication Technology) technologies such as eXtended Reality, which collectively refers to Virtual Reality, Augmented Reality, and Mixed Reality, and Blockchain technology for the security of personal information of users using the control device. However, in this specification, a detailed description of such ICT technologies refers to known technologies, and a separate detailed description is omitted.

Hereinafter, an embodiment of the present disclosure will be described in detail with reference to the attached drawings.

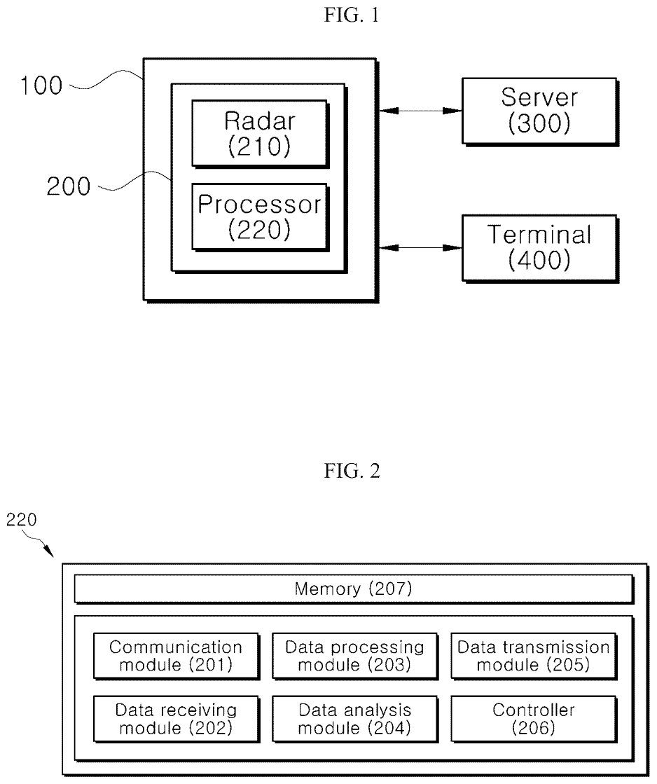

FIG. 1 is a diagram illustrating a system according to an embodiment of the present disclosure.

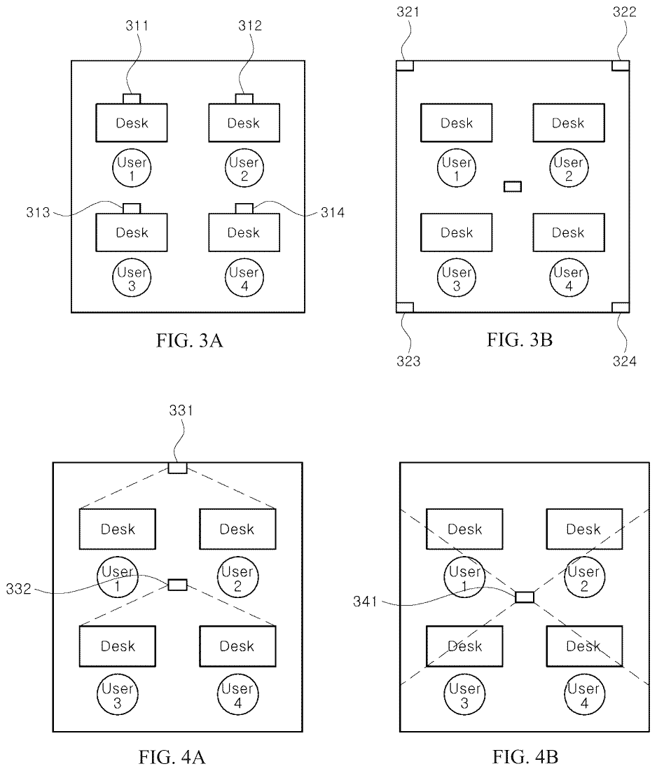

FIG. 2 is a drawing illustrating the processor of FIG. 1.

Referring to FIG. 1, a system 1 for analyzing a work and health status of a user in an office using radar may include an office 100, a work space 200, a server 300, and a terminal 400.

A plurality of the work spaces 200 may be included in the office 100, and a radar 210 and a processor 220 may be provided in the work spaces.

The radar 210 may include at least one radar sensor or radar sensor module. The radar 210 may sense a movement of the user and collect data on the user's breathing, heartbeat, and the like.

The processor 220 may receive and process data for analyzing the work and health status of the user in the office collected by the radar 210. The result of the processing may be transmitted to the server 300 or/and the terminal 400.

Referring to FIG. 2, the processor 220 may include the following components to process data collected by the radar 210.

The processor 220 may include a communication module 201, a data receiving module 202, a data processing module 203, a data analysis module 204, a data transmission module 205, a controller 206, and the like.

The communication module 201 may support a wired/wireless communication protocol for communication with the radar 210 and an external device. Here, the external device may include the server 300 or/and the terminal 400. Meanwhile, the external device may include various devices that store or retain data related to status analysis, and the like in relation to the present disclosure in addition to the above configuration. The wired/wireless communication protocol may include, but is not limited to, Bluetooth, BLE, Zigbee, Wi-Fi, 3G, 4G, 5G, and the like.

The data receiving module 202 may receive data on the user's movement sensed by the radar 210 connected through the communication module 201.

The controller 206 processes only meaningful data related to the user's health status, including the user's breathing and heart status during work, from the user's movement sensing data according to the present disclosure, and removes other noises, collects various data unrelated to the user's movement during work, stores them in advance in the memory 207, and may use them for filtering thereafter.

The data processing module 203 processes the user's movement data collected through the radar 210 received by the data receiving module 202, filters only the data related to the user's breathing and health status during work, and processes the filtered data so that it may be analyzed separately. For convenience, the data related to the health status will be described below using, for example, heart status data as an example.

When the data processing module 203 receives the user's movement data while working from the data receiving module 202, it may call the noise data from the memory 207 and compare it to remove the noise. The data from which the noise is removed may be converted into a frequency domain by processing FFT (Fast Fourier Transform). The data processing module 203 separates the user's movement data while working, which has been converted into a frequency domain by processing FFT, into frequency domain modules, and individually processes the data of each separated region to generate data for analyzing breathing while working and data for analyzing health status. Each analysis data generated in this manner may be directly transferred to the data analysis module 204 or transferred to the data analysis module 204 after processing IFFT (Inverse FFT).

In the above description, when the data processing module 203 receives the user's movement data during work from the data receiving module 202, it first performs FFT processing without filtering it and converts it into a frequency domain, and filters the data converted into the frequency domain to extract only the respiratory activity waveform and heartbeat waveform during work. The filtering may be, for example, one of a low-pass filter, a high-pass filter, or a band-pass filter depending on the noise. The subsequent operation may refer to the above-mentioned content.

The data analysis module 204 may analyze the user's health status by calling and comparing the respiratory activity waveform and heartbeat waveform extracted by the data processing module 203 with the corresponding respiratory activity waveform and heartbeat waveform from the memory 207.

In the data analysis process, the following reference data for analysis may be stored in advance in the memory 207.

The reference data may include, for example, at least one of the following: the respiratory activity waveform and heartbeat waveform of the user during a given period of time; the respiratory activity waveform and heartbeat waveform of the user while working and not working; the respiratory activity waveform and heartbeat waveform at the start and end of work for the user; the respiratory activity waveform and heartbeat waveform of the user according to the entire working hours; the respiratory activity waveform and heartbeat waveform of the user while working and not working according to the day of the week; the respiratory activity waveform and heartbeat waveform of the user while working and not working according to the weather; the respiratory activity waveform and heartbeat waveform of the user according to the time zone; the respiratory activity waveform and heartbeat waveform of the user according to the working posture; the respiratory activity waveform and heartbeat waveform received from an external medical institution database server in each of the above cases; the respiratory activity waveform and heartbeat waveform of a generally healthy person and a person suffering from a given disease from an external medical institution database server; and the like.

The above reference data may have different priorities or weights set during status analysis. The above priorities or weights may also be different depending on the status analysis time. For example, in the case that today is Wednesday and it is raining, the user's work start time is 8 AM, and the total work time is 8 hours, then the reference data corresponding to Wednesday, rain, 8 AM, and 8 hours may be given higher priority or weight. Therefore, when analyzing the user's respiratory activity waveform and heartbeat waveform during work by applying multiple reference data simultaneously or sequentially, the user's health status may be analyzed with reference to the above-mentioned priorities or weights, and the guide including recommended data or content may be provided differently accordingly.

In addition to the user, the above-mentioned reference data may also be collected for other users within a predefined space, other users working at the same workplace, other users of the same gender or age, other users of the same job or position, and the like, and may be used as reference or when analyzing the user's work and health status.

The reference data may include data related to work. At this time, work-related data may include the respiratory activity waveform and heartbeat waveform of a user who is designated as an excellent worker or has received an award, the respiratory activity waveform and heartbeat waveform of a user who is designated as a poor worker or is subject to management, and the respiratory activity waveform and heartbeat waveform of each user belonging to the same work space or the same group. Meanwhile, when collecting work-related data, the predefined time after the start of work and the predefined time before the end of work may not be referred to in the status data analysis. Here, the predefined time may be arbitrarily set as 1 minute, 3 minutes, 5 minutes, 10 minutes, and the like. This is because the predefined time is simultaneous with the start of work or just before the end of work in the corresponding time interval, so it may be judged as the section with the lowest concentration within the corresponding work time.

In addition, the work-related data of other users may be collected and stored by dividing them by time zone, day of the week, weather, and the like. In addition, the work-related data of other users may be stored and used by dividing them by the same gender, same age group, same position or title, and the like.

The reference data may include health-related data. At this time, the health-related data may be collected and stored separately, such as the respiratory activity waveform or heartbeat waveform of a healthy individual with a specific disease or organ based on the result of in-house health checkup, and the respiratory activity waveform or heartbeat waveform of an abnormal health individual with a specific disease or organ problem based on the result of in-house health checkup, and used as reference data.

Meanwhile, the reference data may be stored separately, such as an excellent worker-an excellent health individual, an excellent worker-an excellent health individual, a poor worker-an excellent health individual, a poor worker-an excellent health individual, and the like, by combining and matching the health-related data and the work-related data, and storing and using the respiratory activity waveform and heartbeat waveform separately, such as an excellent worker-an excellent health individual, an excellent workers-an excellent health individual, and a poor worker-an excellent health individual.

The controller 206 monitors a user who is determined to be overworked or poor healthy, continuously collects the respiratory activity waveform and heartbeat waveform data, compares them with the previous respiratory activity waveform and heartbeat waveform, determines whether there is a change in the working status or health status, and regularly creates and provides a report. The information provided in this manner may also provide a standard deviation so that it may be reflected as a weight in the personnel evaluation.

The memory 207 may process and store the critical respiratory activity waveform and heartbeat waveform for health and health abnormalities in advance. Therefore, when the data analysis module 204 first obtains the respiratory activity waveform and heartbeat waveform information of the user while working, it may determine whether to perform secondary processing on the respiratory activity waveform and heartbeat waveform information obtained by applying the critical respiratory activity waveform and heartbeat waveform. For example, when the data analysis module 204 determines that there is a problem and secondary processing is necessary based on the first critical respiratory activity waveform and heartbeat waveform information of the user while working, it may set this as additional health status determination candidate data.

When the data analysis module 204 sets the additional health status determination candidate data, it may skip the primary processing process and perform the secondary processing process directly on the subsequent data. The secondary processing process may refer to comparing and analyzing the respiratory activity waveform and heartbeat waveform more accurately by calling and comparing at least one candidate data from the memory 207 to determine the user's health status. Afterwards, in the case that the data analysis module 204 determines that there is no problem with the health status for a predetermined number of times for example, three times or more, the data analysis module 204 may switch back to the primary processing priority application procedure.

When the data analysis module 204 first performs the secondary processing process, it determines the health status by applying only a predetermined number of the highest priority reference data according to the priority or weight, but in the case that it continues to determine that the health status is abnormal, it may use additional reference data to determine the accurate health status.

In the case that the data analysis module 204 determines that the user's working respiratory activity waveform and heartbeat waveform are continuously determined to be in a healthy state for a predetermined time or a predetermined number of times based on the analysis result, the respiratory activity waveform and heartbeat waveform for a predetermined time or a predetermined number of times may be ignored and not processed. For example, when analyzing a waveform in a unit of time, the data analysis module 204 may skip the waveforms in the next hour the next hour and process the waveforms in the next hour the next hour and two hours after the next hour.

In the above description, when analyzing a waveform in a unit of time, in the case that the analysis result from the waveform for the first 10 minutes is determined to be good health, the analysis term may be increased. For example, the analysis term may be adjusted from analyzing a waveform in a unit of 1 minute to a unit of 5 minutes, 10 minutes, 20 minutes, 30 minutes, and the like. Or, in the case that the waveform analysis result for the first 10 minutes is good, the waveforms for the remaining 50 minutes may not be analyzed.

The data transmission module 205 may generate a signal including recommendation information according to the analysis result and transmit the generated signal.

Here, the recommended information may include, for example, recommended music content, recommended video content, and the like.

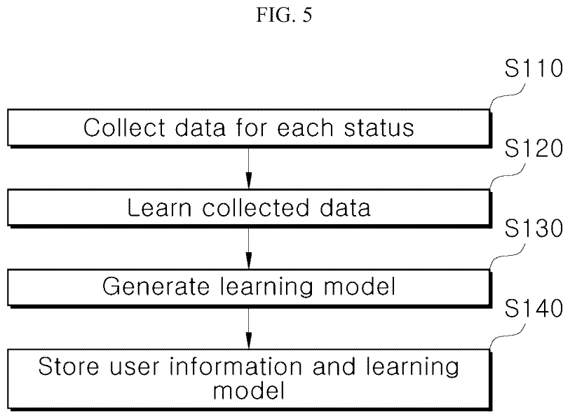

FIGS. 3A to 4B are diagrams illustrating an arrangement of the radar 210 in the workspace 200 in the office 100. This may be for sensing more accurate or less noisy user work movement data through the radar 210.

In FIGS. 3A to 4B, for convenience of explanation, it is assumed that there are four desks and four users (User1-User4) working in the workspace 200.

Referring to FIG. 3A, each of the radars 311-314 may be positioned or arranged on the desk or in front of the desk in the workspace 200, and may ultimately be positioned in front of the user.

On the other hand, in FIG. 3B, each of the radars 321-324 may be placed at a predetermined location within the workspace 200 rather than being placed in front of each desk or user as in FIG. 3A. Here, the predetermined position may include a corner position within the defined workspace 200. Meanwhile, in FIGS. 3A and 3B, the apparatus 325 may be additionally placed at a midpoint of each desk or each user.

Here, the apparatus 325 may additionally include a radar to sense the work status of each user in the same way as each of the radars 321-324.

Alternatively, the apparatus 325 may be a gateway that receives data sensed by each of the radars 321-324 and transmits it to the server 300, or combines it and transmits it to the server 300 or processes it directly.

Referring to FIG. 4A, the work space 200 may be grouped with multiple users, and one radar may be assigned to each group and placed at a predetermined position. In FIG. 4A, two users are grouped, a total of two groups are formed, and two radars 331 and 332 are assigned to each group.

Meanwhile, each of the radars 331 and 332 is illustrated as being placed at a middle position of the users, but is not limited thereto. According to an embodiment, the radars 331 and 332 may be moved in a horizontal or/and vertical direction to more accurately sense the work and health status of the user. For example, it may be moved in front of a first user to sense the work and health status of the first user, and then moved in front of a second user to sense the work and health status of the second user.

In FIG. 4B, the position of each user in the work space 200 may be identified, and a radar 341 may be placed at the midpoint of each user. As described above, in this case, the radar 341 may move in the horizontal and/or vertical directions to sense the user's work and health status from various angles.

In FIG. 2, at least one of the data processing module 203, the data analysis module 204, and the controller 206 may include an artificial intelligence engine AI engine not shown. The artificial intelligence engine may learn using bio-signal data respiratory activity waveform, heartbeat waveform, and the like collected from the user or an external device as a training data set, and generate a learning model. The learning model generated in this way may function to replace some operations of the data processing module 203 or/and the data analysis module 204 described above.

The server 300 may be located in-house or remotely, and may communicate with components of the workspace 200 based on an intranet or the internet to exchange data.

The server 300 may replace all or at least part of the functions of the processor 220 included in the workspace 200.

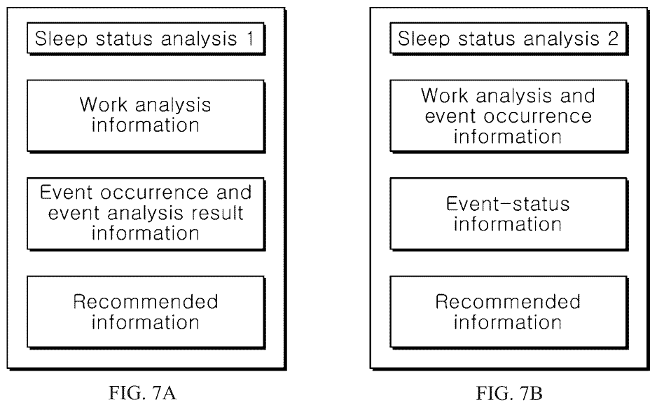

The terminal 400 may communicate with the processor 220 of the workspace 200 and/or the server 300, and may receive and output the result of analyzing data on the user's work and health status collected through the radar of the workspace 200 while working. Here, the results may also include guide data such as health recommendation based on the analysis, such as in FIG. 7A or 7B, for example.

The terminal 400 may be a terminal held by the user, a terminal placed on a desk placed in the workspace 200, or a terminal registered to the server 300. This terminal 400 may be a fixed terminal type such as a PC or TV, or a mobile terminal type such as a smartphone, tablet PC, or laptop.

The terminal 400 may also manually control each radar in the workspace 200 to collect data for analyzing the user's work status while working. At this time, the signal regarding control may be transmitted through the server 300 or directly to the radar.

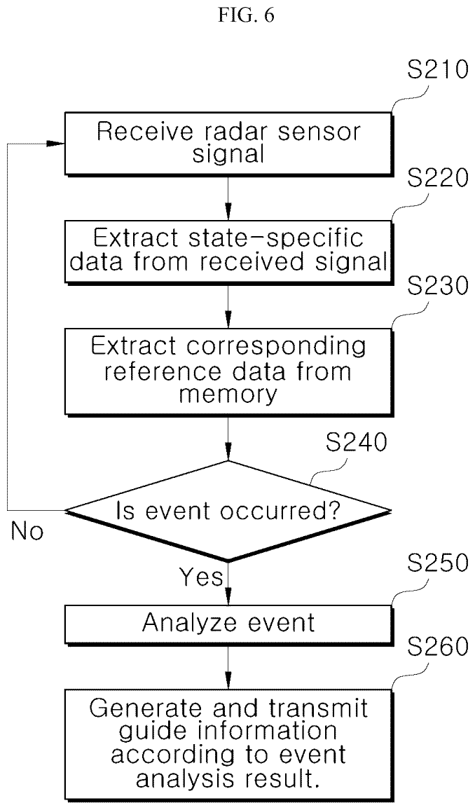

FIGS. 5 and 6 are flowcharts illustrating a method for analyzing a user's work status using radar according to an embodiment of the present disclosure.

In this specification, the status while the user is working may include a health status, a work status, and the like.

In an electronic apparatus according to at least one of various embodiments of the present disclosure, a method for analyzing a work and health status using radar placed in front of each user may include storing reference data; obtaining movement data of a user while working using the radar; converting the obtained movement data of the user while working into a frequency domain and obtaining respiratory activity waveform and heartbeat waveform data; analyzing the obtained respiratory and heartbeat waveform data by comparing the data with the stored reference data; and generating and transmitting guide data including recommended content based on the analysis result.

Referring to FIG. 5, in operation step S110, the processor 220 may collect the user's work and health status data. Here, the status data may include work status data, health status data, and the like.

In operation step S120, the processor 220 may learn the collected data.

In operation step S130, the processor 220 may perform learning for analyzing the user's work status and may generate an artificial intelligence model.

In operation step S140, the processor 220 may store the user information and the learning model in the memory 207.

Referring to FIG. 6, in operation step S210, the processor 220 may receive a radar sensor signal.

In operation step S220, the processor 220 may extract state-specific respiration and heartbeat movement data, that is, the respiration activity waveform and heartbeat waveform, from the radar sensor signal.

In operation step S230, the processor 220 may extract corresponding reference data from the memory 207.

In operation step S240, the processor 220 may compare the two data described above to determine whether an event is occurred. Here, the event may indicate a predefined status. The predefined state may include abnormal health, good health, health risk, health risk alert, rest, work, and the like. The predefined status may also include fatigue, stress, drowsiness, and the like.

In operation step S250, in the case that the processor 220 determines that an event is occurred as a result of the determination of operation step S240, the processor 220 may analyze the event.

In operation step S260, the processor 220 may generate and provide a guide such as FIGS. 7A and 7B according to the event analysis result.

The guide may include content or data such as a rest recommendation, a work recommendation, an exercise guide at the desk, and a sound guide for stress and relaxation.

According to the present disclosure described above, there is an advantage in that the work and health status of each user working in the office may be more accurately identified and analyzed, and various events that may occur may be appropriately responded to.

The steps of the method or algorithm described in relation to the embodiments of the present disclosure may be implemented directly in hardware, implemented as a software module executed by hardware, or implemented by a combination of these. The software module may reside in a random access memory (RAM), a read only memory (ROM), an erasable programmable ROM (EPROM), an electrically erasable programmable ROM (EEPROM), a flash memory, a hard disk, a removable disk, a CD-ROM, or any other form of computer readable recording medium well known in the art to which the present disclosure pertains.

Although the embodiments of the present disclosure have been described above with reference to the attached drawings, those skilled in the art will understand that the present disclosure may be implemented in other specific forms without changing the technical spirit or essential characteristics thereof. Therefore, it should be understood that the embodiments described above are exemplary in all respects and not restrictive.

What is claimed is:

1. An apparatus for analyzing a work and health status using radar, comprising:
a memory;
a radar sensor positioned in front of a user to sense movement data of the user while working; and
a processor configured to analyze the work and health status by obtaining data sensed by the radar sensor,
wherein the processor is configured to convert the data sensed by the radar sensor into a frequency domain and obtain respiratory activity waveform and heartbeat waveform data.

2. The apparatus according to claim 1, wherein the processor is configured to:
filter the data sensed by the radar sensor converted into the frequency domain and obtain the respiratory activity waveform and heartbeat waveform data.

3. The apparatus according to claim 2, wherein the filtering is performed by a band-pass filter for a frequency domain corresponding to the respiratory activity waveform and heartbeat waveform data.

4. The apparatus according to claim 3, wherein the processor is configured to:
read out reference data for comparison with the respiratory activity waveform and heartbeat waveform data obtained from the memory.

5. The apparatus according to claim 3, wherein the reference data includes reference data for determining the work status of the user and reference data for determining the health status of the user.

6. The apparatus according to claim 5, wherein the reference data further includes matching data of the reference data for determining the work status of the user and the reference data for determining the health status of the user.

7. The apparatus according to claim 6, wherein the reference data includes:
reference data for determining the work status including the respiratory activity waveform and heartbeat waveform of the user who is designated as an excellent worker or is received an award, the respiratory activity waveform and heartbeat waveform of the user who is designated as a poor worker or is subject to management, the respiratory activity waveform and heartbeat waveform of each user belonging to a same work space or a same group, and
reference data for determining the health status including the respiratory activity waveform and heartbeat waveform of the user with excellent health in relation to a specific disease or organ based on a result of an in-house health checkup, and the respiratory activity waveform or heartbeat waveform of the user with abnormal health in relation to a specific disease or organ based on a result of an in-house health checkup.

8. The apparatus according to claim 7, wherein a priority and a weight of each of the reference data are different from each other, and
wherein the priority and the weight of each of the reference data are different depending on a time of the work and health status analysis.

9. The apparatus according to claim 8, wherein the processor is configured to:
control to generate and transmit guide data including recommended data and content based on the work and health status analysis result.

10. A method for analyzing a work and health status using radar disposed in front of each user, the method performed by a processor of an electronic apparatus comprising:
storing reference data;
obtaining movement data of a user while working using the radar;
converting the obtained movement data of the user while working into a frequency domain and obtaining respiratory activity waveform and heartbeat waveform data;
analyzing the obtained respiratory and heartbeat waveform data by comparing the data with the stored reference data; and
generating and transmitting guide data including recommended content based on the analysis result.

11. The method according to claim 10, further comprising:
filtering the data sensed by the radar sensor converted into the frequency domain and obtain the respiratory activity waveform and heartbeat waveform data.

12. The method according to claim 11, wherein the filtering is performed by a band-pass filter for a frequency domain corresponding to the respiratory activity waveform and heartbeat waveform data.

13. The method according to claim 12, further comprising:
reading out reference data for comparison with the respiratory activity waveform and heartbeat waveform data obtained from the memory.

14. The method according to claim 13, wherein the reference data includes reference data for determining the work status of the user and reference data for determining the health status of the user.

15. The method according to claim 14, wherein the reference data further includes matching data of the reference data for determining the work status of the user and the reference data for determining the health status of the user.

16. The method according to claim 15, wherein the reference data includes
reference data for determining the work status including the respiratory activity waveform and heartbeat waveform of the user who is designated as an excellent worker or is received an award, the respiratory activity waveform and heartbeat waveform of the user who is designated as a poor worker or is subject to management, the respiratory activity waveform and heartbeat waveform of each user belonging to a same work space or a same group, and
reference data for determining the health status including the respiratory activity waveform and heartbeat waveform of the user with excellent health in relation to a specific disease or organ based on a result of an in-house health checkup, and the respiratory activity waveform or heartbeat waveform of the user with abnormal health in relation to a specific disease or organ based on a result of an in-house health checkup.

17. The method according to claim 16, wherein a priority and a weight of each of the reference data are different from each other, and
wherein the priority and the weight of each of the reference data are different depending on a time of the work and health status analysis.

18. The method according to claim 17, further comprising:
controlling to generate and transmit guide data including recommended data and content based on the work and health status analysis result.
